(19) United States
(12) Patent Application Publication
(10) Pub. No.: US 2025/0134131 A1
(43) Pub. Date: May 1, 2025

(54) A BATCH PROCESS AND SYSTEM FOR PREPARING A LIQUID OAT BASE OR DRINK FROM AN OAT MATERIAL

(71) Applicant: OATLY AB, MALMÖ (SE)

(72) Inventors: David HELLBORG, MALMÖ (SE); Joakim BJUNÖ, MALMÖ (SE); Karin PETERSSON, MALMÖ (SE); Sofia EHLDE, MALMÖ (SE)

(21) Appl. No.: 18/726,055

(22) PCT Filed: Dec. 30, 2021
(86) PCT No.: PCT/EP2021/087833

§ 371 Date: Jul. 1, 2024

(51) Int. Cl. A23J 1/12; A23J 3/14; C12N 9/26; C12N 9/80

(52) U.S. Cl. CPC A23J 1/125; A23J 3/14; C12N 9/2411; C12N 9/80

(57) ABSTRACT

The invention relates to a process preparing a liquid oat base or drink from an oat derived material. The process comprises filling a first enzymation tank with the oat derived material and an enzyme, enzymating the oat derived material in the first enzymation tank, filling a second enzymation tank with the oat derived material and an enzyme, the second enzymation tank being fluidly connected in parallel with the first enzymation tank, enzymating the oat derived material in the second enzymation tank, producing a continuous flow of enzymised oat derived material by alternately emptying the first and second enzymation tanks, and heating, by a first heating device fluidly connected to and arranged downstream of the first and second enzymation tanks, the continuous flow of enzymised oat derived material to terminate the enzymation thereof. The invention also relates to a system for preparing a liquid oat base or drink from an oat derived material.

### TECHNICAL FIELD OF INVENTION

The invention relates to a process and system for preparing a liquid oat base or drink from an oat material.

### BACKGROUND

There is a growing interest in refraining from consumption of dairy products and from meat products, for example for health reasons, religious reasons, and sustainability reasons. In addition, there is a growing trend to choose products for consumption in the daily life which contribute to a positive climate impact. This may mean excluding or at least reducing intake of dairy based products as well as meat products.

Oat is well known for containing nutritious compounds and may be considered a sustainable vegetable ingredient in food production. Oat in different forms is therefore an often-used ingredient in healthy and nutritious foods.

For example, use of oats in production of oat-containing aqueous liquid, sometimes called an oat base, is well known. Such oat base may be used as ingredient or base for manufacturing of different food products, for example, analogues for dairy products. Oat beverage, such as an oat drink is one well known example.

Manufacturing of oat base, and oat drink, typically involves subjecting micronized oats to one starch degrading enzymatic treatment in aqueous suspension. Process parameters, such as temperature, have an effect on both enzymes and the micronized oats of the suspension. It is challenging and problematic during the manufacturing to maintain the micronized oats in the aqueous suspension, in a form suitable for the enzymatic treatment. It is further problematic to control the enzymatic activities, as well as maintaining nutritious qualities from the oats, during the manufacturing process.

There is, thus, a need for providing improved control of manufacturing process for the oat base, while at the same maintaining control of the quality of the oat base.

### SUMMARY OF INVENTION

It is an objective of the present invention to, at least partly, improve the overall process of preparing an oat base or oat drink.

According to a first aspect of the invention, these and other objects are achieved, in full or at least in part, by a process for preparing a liquid oat base or drink from an oat derived material. The process comprises filling a first enzymation tank with the oat derived material and an enzyme, enzymating the oat derived material in the first enzymation tank, filling a second enzymation tank with the oat derived material and an enzyme, the second enzymation tank being fluidly connected in parallel with the first enzymation tank, enzymating the oat derived material in the second enzymation tank, producing a continuous flow of enzymised oat derived material by alternately emptying the first and second enzymation tanks, and heating, by a first heating device fluidly connected to and arranged downstream of the first and second enzymation tanks, the continuous flow of enzymised oat derived material to terminate the enzymation thereof.

This is advantageous in that the process can run uninterruptedly by switching between the two enzymation tanks. This provides for major cost-savings since the process will be automized and continuous.

The process may further comprise cooling the enzymised oat derived material after heating the continuous flow of enzymised oat derived material.

The process may further comprise steaming the enzymised oat derived material after the step of heating the enzymised oat derived material to terminate the enzymation and before the step of cooling the enzymised oat derived material.

The process may further comprise filling a third enzymation tank with the enzymised oat derived material and enzymes, enzymating the enzymised oat derived material in the third enzymation tank, filling a fourth enzymation tank with the enzymised oat derived material and enzymes, the fourth enzymation tank being fluidly connected in parallel with the third enzymation tank, enzymating the enzymated oat derived material in the fourth enzymation tank, and producing a continuous flow of enzymised oat derived material by alternately emptying the third and fourth enzymation tanks.

In other words, the oat derived material may be subjected to two steps of enzymation, the first in either the first enzymation tank or the second enzymation tank, and the second in the third enzymation tank or the fourth enzymation tank.

The enzymation of the oat derived material in the first or second enzymation tank may be a first enzymation of oat derived material in aqueous suspension, wherein a first enzymatically treated suspension is formed.

The heating the oat derived material from the enzymation in the first or second enzymation tank to terminate the enzymation thereof, may be heating the first enzymatically treated suspension, wherein a heat-treated suspension is formed.

The enzymation in the third or fourth enzymation tank of the oat derived material from the heating may be a second enzymation of the heat-treated suspension, wherein a second enzymatically treated suspension is formed. The second enzymatically treated suspension, may, with or without further treatment, be an oat base or drink.

Thus, the enzymation in the third or fourth enzymation tank may, optionally, be followed by further treatment. The further treatment may be, for example, removal of particulate matter or compounds, for example fiber residues or other matter originating from the oats. For example, the further treatment may be sedimentation, decantation, or filtering.

The process may further comprise heating, by a second heating device fluidly connected to and arranged downstream of the third and fourth enzymation tanks, the continuous flow of enzymised oat derived material a second time to terminate the enzymation thereof.

The process may further comprise cooling the enzymised oat derived material a second time after heating the continuous flow of enzymised oat derived material the second time.

The process may further comprise steaming the enzymised oat derived material a second time after the step of heating the enzymised oat derived material a second time to terminate the enzymation and before the step of cooling the enzymised oat derived material a second time.

The oat derived material may comprise micronized oats or micronized oat-kernels, or oat-flour, or fractions of oat. For example, the oat derived material may, thus, be in form of milled, crushed or ground oat derived material, or a combination thereof. As another example, the fractions of oat may be oat bran or fiber reduced oat flour.

The oat derived material may be a mixture of oat and water. For example, the oat derived material may be a mixture of oat and water an aqueous or water-based suspension or slurry of one or more of micronized oat-kernels, oat-flour, and fractions of oat.

If the oat derived material comprises or is in form of micronized oats or micronized oat-kernels, or oat-flour, or fractions of oat, the oat derived material may be oat derived material provided for or entering the first enzymation. Such oats, for example micronized oats, will in the process be at least partially degraded or transformed, for example by means of the first and second enzymation, and it shall be understood that as it progresses in the process it may cease to be in the provided form of micronized oats or oat-kernels.

The oat derived material may be provided as an aqueous or water suspension or slurry.

The step of enzymating in the first and second enzymation tanks and/or in the third and fourth enzymation tanks may comprise contacting the oat derived material with starch degrading enzymes, preferably amylase.

The step of enzymating in the first and second enzymation tanks and/or in the third and fourth enzymation tanks may comprise contacting the oat derived material with protein solubilizing enzyme, preferably protein deamidase or protein glutaminase.

Thereby, the liquid oat base or drink having an improved soluble oat protein content may be provided. A desirable and synergistic effect, which improves levels of solubilized proteins, is achievable from the combination of using starch degrading enzymes and protein solubilizing enzymes. Enzymation of the product in two separate steps will make it possible to provide a product with a more even quality.

After the step of enzymation in the first or second enzymation tanks the oats may be subjected to higher temperatures of the heating. This is believed to be, at least in part, a result of starch degradation in the first step of enzymation, which results in lower degree of gelatinization during the heat treatment as compared to heat treatment without a preceding first step of enzymation.

Other enzymes for solubilizing proteins may be used than the protein deamidase or protein glutaminase. Solubilization of proteins, as used herein, is intended to describe an increase of solubility of the proteins in water or aqueous liquid as compared to prior to or without the treatment, such that at least a portion of the proteins present in the oat derived material may be provided in aqueous or water solution. Solubilization may be realized, for example, by enzymes modifying amino acids of the proteins, such as side chains of amino acids, into more hydrophilic form, for example by converting amide groups to the side chains. Any type of enzymes suitable for this purpose in providing an oat base or oat drink may be considered for the purpose. Converting the proteins into a more water-soluble form, or solubilizing proteins, allows for more efficient process for preparing a liquid oat base or drink from an oat derived material, as well as improved properties of the oat base or drink. Increase solubility may, for example, improve extraction of proteins from an oat derived material matrix during the process, thus increasing levels of oat protein in the oat base or drink.

According to a second aspect of the invention, these, and other objects, and/or advantages that will be apparent from the following description, are achieved, in full or at least in part, by a system for preparing a liquid oat base or drink from an oat derived material. The system comprises a first enzymation section adapted for a first enzymation of the oat derived material, and a first heating device fluidly connected to and arranged downstream of the first enzymation section and adapted to heat the oat derived material from the first enzymation, wherein the first enzymation section comprises at least two enzymation tanks fluidly connected in parallel, the at least two enzymation tanks being configured to produce a continuous flow of enzymised oat derived material.

The heating device may be adapted to both heat the enzymised oat derived material in a first step and thereafter cool the enzymised oat derived material in a second step.

The heating device may comprise a heat exchanger and/or a steam injector. oat derived material

The system further comprise a second enzymation section adapted for a second enzymation of the oat derived material and fluidly connected downstream to the first heating device, and a second heating device fluidly connected to and arranged downstream of the second enzymation section and adapted to heat the oat derived material from the second enzymation, wherein the second enzymation section comprises at least two enzymation tanks fluidly connected in parallel, the at least two enzymation tanks being configured to produce a continuous flow of enzymised oat derived material.

The second heating device may be adapted to both heat the enzymised oat derived material in a first step and thereafter cool the enzymised oat derived material in a second step.

The heating device may comprise a heat exchanger and/or a steam injector. oat derived material

The first enzymation section and the second enzymation section may comprise a conduit or a reservoir, such as a tank, respectively.

Effects and features of the second aspect of the present invention are largely analogous to those described above in connection with the first aspect of the inventive concept. Examples mentioned in relation to the first aspect of the present invention are largely compatible with the further aspects of the invention.

Other objectives, features and advantages of the present invention will appear from the following detailed disclosure, from the attached claims, as well as from the drawings. It is noted that the invention relates to all possible combinations of features.

Generally, all terms used in the claims are to be interpreted according to their ordinary meaning in the technical field, unless explicitly defined otherwise herein. All references to “a/an/the [element, device, component, means, step, etc.]” are to be interpreted openly as referring to at least one instance of the element, device, component, means, step, etc., unless explicitly stated otherwise.

The oat base or drink material may comprise, by dry weight, 5-20% protein, and 3-15% fat, and optionally 10-80% carbohydrates, including sugars. Typically, the oat base or oat drink may comprise sugars 2-8%, fibers 0.2-3% (out of which 0.1-1% of the oat drink are beta glucans), fat 0.0-2.5%, and proteins 0.5-2.5%, by weight of the oat base or drink. The liquid oat base or drink may comprise an oat derived material with 5-20% by weight dry substance, based on the weight of the liquid oat base or drink.

Herein is disclosed a process for preparing the liquid oat base or drink from the oat derived material. It shall be understood that the oat derived material changes in the process. In the present disclosure, in an attempt to improve understanding and clarity of the disclosure, the term oat derived material is sometimes used to describe and refer to oat derived material having undergone different treatment in the process, and, thus, one oat derived material may not be the same as another oat derived material. Sometimes herein, if deemed to improve clarity, the term oat derived material is further specified, for example, by defining it as an oat derived material from the first enzymation, or an oat derived material from the heating etc. Some examples of changes, which the oat derived material may undergo in the process, will now be given. Oat derived material in the first enzymation and the second enzymation may, for example, be subjected to starch degrading enzymes, which enzymes may degrade starch present in the oat derived material and, thus, change the oat derived material. As a further example, heating the oat derived material may involve swelling of starch in the oat derived material, thus changing the oat derived material. To further exemplify, oat derived material early in the first enzymation may be micronized oat kernels with essentially intact starch, while oat derived material at the end of the first enzymation may be oat derived material comprising degraded starch.

The oat base or drink from the process may be mixed with additional additives and may then be referred to a liquid oat composition.

Herein is disclosed a process for preparing the liquid oat base or drink from the oat derived material by contacting the oat derived material with the enzyme such as the starch degrading enzyme, and the protein solubilizing enzyme. It shall be understood that the contact may be performed in many different ways such as for example by mixing the oat derived material and the enzyme in a supply or vessel such as a tank and/or a pipe. The mixing may be performed by any known mixing techniques such as in a mixing tank comprising impellers or blades.

As used herein, the term “comprising”, and variations of that term are not intended to exclude other additives, components, integers, or steps.

### BRIEF DESCRIPTION OF THE DRAWINGS

The above, as well as additional objects, features, and advantages of the present invention, will be better understood through the following illustrative and non-limiting detailed description of the present invention, with reference to the appended drawings, where the same reference numerals may be used for similar elements, and wherein:

FIGS. 1A and 1B are flow charts of two exemplary processes for preparing a liquid oat base or drink from an oat derived material.

FIG. 2 is a schematic view of an example of a system for preparing a liquid oat base or drink from an oat derived material.

FIG. 3 is a schematic view of an example of a system for preparing a liquid oat base or drink from an oat derived material.

FIG. 4 is a schematic view of a heating device in a system for preparing a liquid oat base or drink from an oat derived material.

FIG. 5a to FIG. 5c are schematic views of examples of systems for preparing a liquid oat base or drink from an oat derived material.

### DETAILED DESCRIPTION

The present inventive concept will now be described more fully hereinafter with reference to the accompanying drawings, in which currently preferred variants of the inventive concept are shown. This inventive concept may, however, be implemented in many different forms and should not be construed as limited to the variants set forth herein; rather, these variants are provided for thoroughness and completeness, and fully convey the scope of the present inventive concept to the skilled person.

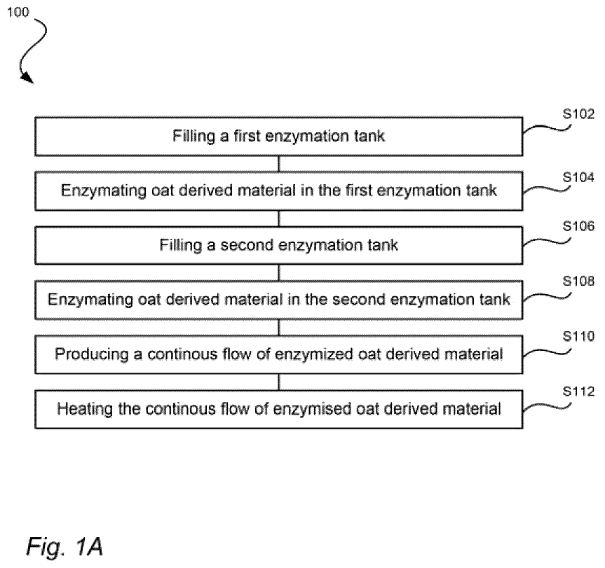

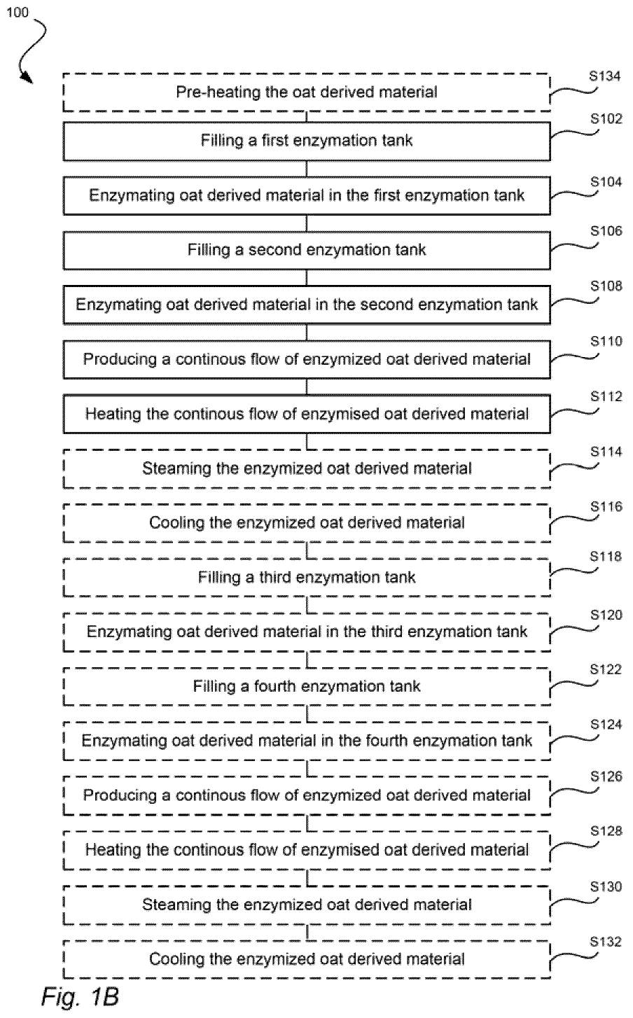

FIG. 1A is a flow chart illustrating steps of a process for preparing a liquid oat base or drink from an oat derived material. Below, the different steps are described in more detail. Even though illustrated in a specific order, the steps of the process 100 may be performed in any suitable order, as well as multiple times and in parallel.

A first enzymation tank is filled S102 with the oat derived material and an enzyme. The oat derived material may comprise micronized oats or micronized oat-kernels, or oat-flour. The oat derived material may be provided as an aqueous or water suspension or slurry. The oat derived material in the first enzymation tank is enzymated S104 for a desired time, such as for example 30-90 minutes, preferably, 50-70 minutes.

A second enzymation tank is filled S106 with the oat derived material and an enzyme. The second enzymation tank being fluidly connected in parallel with the first enzymation tank. The oat derived material in the second enzymation tank is enzymated S108 for a desired time, such as for example 30-90 minutes, preferably, 50-70 minutes.

The filling of the oat derived material and the enzyme in the tanks S102, S106 may be performed simultaneously or at least partly simultaneously. Thus, the oat derived material and the enzyme may be filled into the tank during a same time-period, or for example filling the tank with a desired amount of the oat derived material takes a longer time than adding a desired amount of the enzyme to the tank.

The first and second enzymation S104, S108 may comprise contacting the oat derived material with a starch degrading enzyme. The starch degrading enzyme may preferably be amylase. Alternatively, or in addition, the first and second enzymation S104, S108 may comprise contacting the oat derived material with a protein solubilizing enzyme. The protein solubilizing enzyme may preferably be a protein deamidase such as protein glutaminase.

Following, by alternatively emptying the first and second enzymation tanks a continuous flow of enzymised oat derived material is produced S110. More specifically, the steps of filling S102, S106 and enzymating in one of the enzymation tanks S104, S108 may be performed while the other tank is emptied of enzymised oat derived material and vice versa. Enzymised oat derived material may be interpreted as oat derived material which has been subject to any degree of enzymation.

The continuous flow of enzymised oat derived material is heated S112 by a first heating device. The first heating device is fluidly connected to and arranged downstream of the first and second enzymation tanks. The continuous flow of enzymised oat derived material may be heated S112 to terminate the enzymation thereof. The enzymation may be either completely or partly terminated. The heating process S112 may have the effect of making the oat derived material more susceptible to the enzymes. In other words, the oat derived material may be more accessible for enzymation.

For example, the heating of the oat derived material may gelatinize starch present in the oat derived material and allow swelling of starch and swelling of any present oat derived material granules, thereby providing open oat derived material. Open oat derived material may also be referred to as open oat particles, swelled starch, or hydrated starch.

It shall be understood that as oat derived material is treated in the process 100, for example by enzymes acting on the oat derived material, or the oat derived material being heated, the oat derived material typically changes, for example resulting from depletion of starch from the oat derived material. Oat derived material having undergone, for example the enzymation in the first enzymation tank S104, may therefore, herein, be referred to as oat derived material from the first enzymation tank, to differentiate it from e.g., oat derived material provided before the enzymation in the first enzymation tank S104. Oat derived material which has been subject to any degree of enzymation may alternatively be referred to as enzymised oat derived material.

FIG. 1B is a flow chart illustrating the steps of the process for preparing the liquid oat base or drink from the oat derived material as described above in connection with FIG. 1A but with some optional additional steps. Below, the different steps are described in more detail. Even though illustrated in a specific order, the steps of the process 100 may be performed in any suitable order, as well as multiple times and in parallel.

The enzymised oat derived material may be cooled S116. The cooling S116 of the enzymised oat derived material may be performed after heating S112 the continuous flow of enzymised oat derived material.

The enzymized oat derived material may be steamed S114. In other words, the enzymised oat derived material may be injected with steam to increase the heat of the oat derived material. Thus, further terminating the enzymation. The steaming S114 of the enzymised oat derived material may be performed after the heating S112 of the enzymised oat derived material. The steaming S114 of the enzymised oat derived material may be performed before the cooling S116 of the enzymised oat derived material.

A third enzymation tank may be filled S118 with the enzymised oat derived material from the heating S112 or steaming S114. The third enzymation tank is arranged downstream of the first heating device. The enzymised oat derived material is thus the oat derived material which has been enzymised in the first or second enzymation tank S104, S108 and then heated S112 and/or steamed S114.

The enzymised oat derived material in the third enzymation tank is enzymised S120 for a desired time, such as for example 30-90 minutes, preferably, 50-70 minutes.

A fourth enzymation tank may be filled S122 with the enzymised oat derived material.

The enzymised oat derived material in the fourth enzymation tank may be enzymised S124 for a desired time, such as for example 30-90 minutes, preferably, 50-70 minutes.

The enzymation of the third and fourth enzymation tank S120, S124 may comprise contacting the oat derived material with a starch degrading enzyme. The starch degrading enzymes may preferably be amylase. Alternatively, or in addition, the enzymation in the third and fourth enzymation tank may comprise contacting the oat derived material with a protein solubilizing enzyme. The protein solubilizing enzyme may preferably be protein deamidase such as protein glutaminase.

The enzymation in the first and second enzymation tanks S104, S108 may be performed in the same way as the enzymation in the third and fourth enzymation tanks S120, S124. For example, both the first and second enzymation S104, S108 and the third and fourth enzymation S120, S124 may comprise using starch degrading enzymes. Alternatively, both the first and second enzymation S104, S108 and the third and fourth enzymation S120, S124 may comprise using protein solubilizing enzymes. Even though the same enzymes may be used, a ratio between oat derived material and enzyme of the respective enzymation may be different. For example, more enzyme may be added in the first and second enzymation S104, S108 than the third and fourth enzymation S120, S124 or the opposite. Alternatively, different amounts of enzyme are used in the respective enzymation for the first, second, third and fourth enzymation S104, S108, S120, S124.

Similarly, a time period in which the oat derived material is enzymised in the first and second enzymation S104, S108 and the third and fourth enzymation S120, S124 respectively may be different.

As another example, the enzymation in the first and second enzymations S104, S108 and/or the enzymation in the third and fourth enzymations S120, S124 may comprise using both the starch degrading enzymes and the protein solubilizing enzymes.

The first and second enzymation S104, S108 and the third and fourth enzymation S120, S124 may be performed in different ways. For example, the first and second enzymation S104, S108 may comprise using starch degrading enzymes while the third and fourth enzymation S120, S124 may comprise using protein solubilizing enzymes. Further, the ratio between enzymes and oat derived material in the respective enzymation may be different, as well as the time-period in which the oat derived material is enzymised.

A continuous flow of enzymised oat derived material may be produced S126 by alternately emptying the third and fourth enzymation tanks.

The continuous flow of enzymised oat derived material may be heated S128 by a second heating device. The continuous flow of enzymised oat derived material may be heated S128 to terminate the enzymation thereof. The enzymation may be entirely terminated or only partly terminated. The second heating device is fluidly connected to and arranged downstream of the third and fourth enzymation tanks.

The enzymised oat derived material may be cooled S132. The cooling S132 of the enzymised oat derived material may be performed after heating S128 the continuous flow of enzymised oat derived material.

The enzymized oat derived material may be steamed a second time S130. The second steaming S130 of the enzymised oat derived material may be performed after the heating S128 of the enzymised oat derived material and before the second cooling S132 of the enzymised oat derived material.

Thus, the heating S112, S128 and/or steaming S114, S130 and/or cooling S116, S132 of the oat derived material may be performed at the first enzymation S104,108, the second enzymation S120,124 or at both the first enzymation S104,108 and the second enzymation S120,124. The heating S112, S128 and/or steaming S114, S130 may be performed at a same temperature or at different temperatures. In some examples the temperature is chosen based on a desired temperature to deactivate enzymation partly or fully.

In an example, the oat derived material may be heated in a pre-heating step S134, before filling S102 the first enzymation tank. The heat treatment of the oat derived material in the pre-heating step S134 may be milder than the first and/or second heating S112, S128. In other words, the oat derived material may be heated to a lower temperature during the pre-heating S134, than during the first and/or second heating S112, S128.

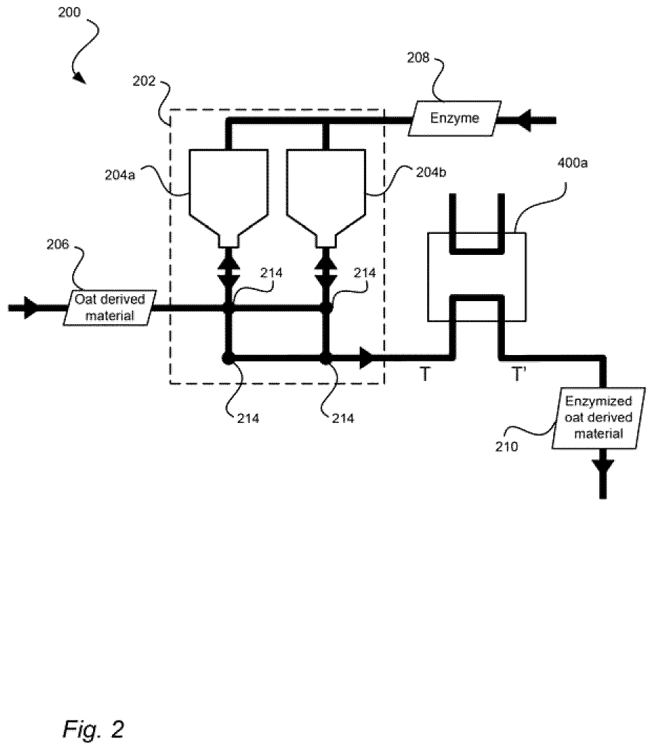

FIG. 2 schematically illustrates one example of a system 200 for preparing a liquid oat base or drink from an oat derived material.

The system 200 comprises a first enzymation section 202. The first enzymation section 202 is adapted for the first enzymation of the oat derived material S104, S108. The first enzymation section 202 may thus be configured to receive the oat derived material 206 and the enzyme 208. Put differently, the first enzymation section 202 may be fluidly connected to a supply (not shown) of oat derived material. Further, the first enzymation section 202 may be fluidly connected to a supply (not shown) of enzyme. The enzyme 208 may be added to the oat derived material 206 prior to entering the first enzymation section 202. Thus, the first enzymation section 202 may have only one inlet/outlet.

The first enzymation section 202 comprises two enzymation tanks 204. The first enzymation section 202 may comprise more than two enzymation tanks 204, such as three, four or more. The number of tanks 204 may be selected based on a desired throughput, redundancy, factory space, pipe size, tank size and so on. For example, having more enzymation tanks 204 may allow for having tanks 204 with smaller volumes as well as shorter enzymation times. A possible associated advantage may be that if the tank 204 or the enzymation process in the tank 204 would fail, the downtime is shorter since the time until the next tank 204 with enzymised oat derived material is finished is shorter. Having fewer enzymation tanks 204 may be advantageous in that less floor space is required, less installed equipment and less maintenance.

The at least two enzymation tanks 204 is configured to produce a continuous flow of enzymised oat derived material 210. The at least two enzymation tanks 204 is fluidly connected in parallel. In the illustration in FIG. 2, the two enzymation tanks 204 are connected by connection points 214 which may comprise valves or other regulators to control for example a flow rate and direction of flow, such that one of the enzymation tanks 204 may be emptied, while the other enzymation tank 204 is filled with the oat derived material 206 and the enzyme 208 or while the enzymation is taking place. More specifically, the enzymation tanks 204a, 204b may each have a volume V, a filling time t1 and an emptying time t2. The enzymation tank 204a, 204b with volume V may have an enzymation time te. The total time for filling t1, enzymating te and emptying t2 one enzymation tank may be approximately one hour. A continuous outflow of enzymised oat derived material 210 can thus be achieved by setting the emptying time t2 equal to the filling time t1 and enzymation time te together.

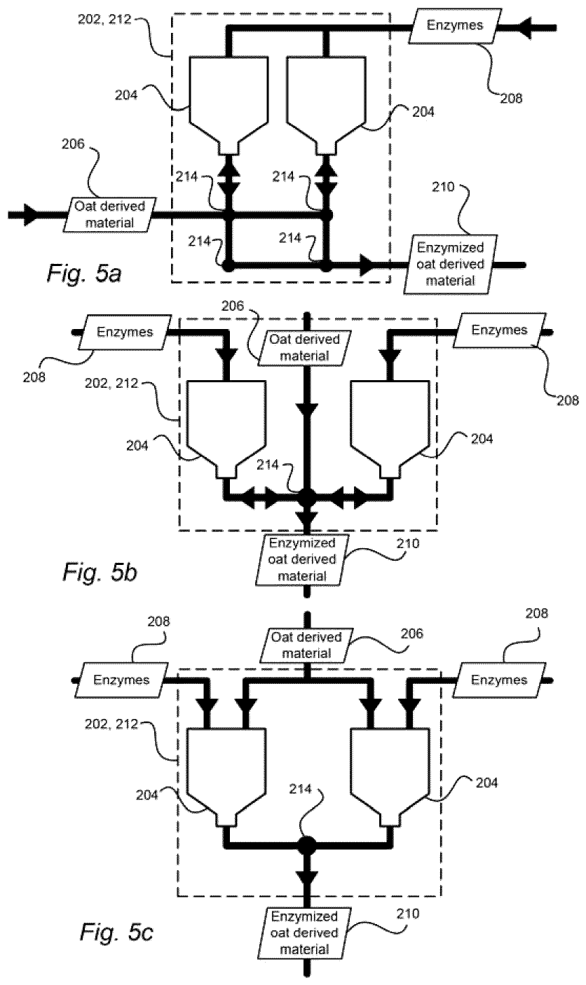

Thus, one of the at least two enzymation tanks 204a, 204b can be emptied while the other one of the at least two enzymation tanks 204a, 204b are filled with new oat derived material 206 and the enzyme 208 such that the oat derived material 206 is filled in the enzymation tank 204a.204b and enzymated. When the first enzymation tank 204a, 204b has been emptied, the two enzymation tanks 204a, 204b can switch so that the now empty enzymation tank 204a, 204b is filled with new oat derived material 206 to be enzymised while the other enzymation tank 204a, 204b is being emptied. The volume V of the enzymation tanks 204a, 204b of the first enzymation section 202 may be 6 000-18 000 L. The connection between the at least two enzymation tanks 204 is further discussed in connection with FIG. 5.

The filing of the tanks 204a, 204b with oat derived material 206 and enzyme 208 for enzymation may be performed simultaneously, or at least partly simultaneal. The filing of the tanks 204a, 204b with oat derived material 206 and enzyme 208 for enzymation may also be performed sequentially, for example by first filing with oat derived material 206 and then with enzyme 208. The tanks 204 may also be used as buffers if there is any disturbance or error downstream of the tanks 204. The tanks 204 may then prolong the enzymation or slow down the filing until the downstream error has been resolved.

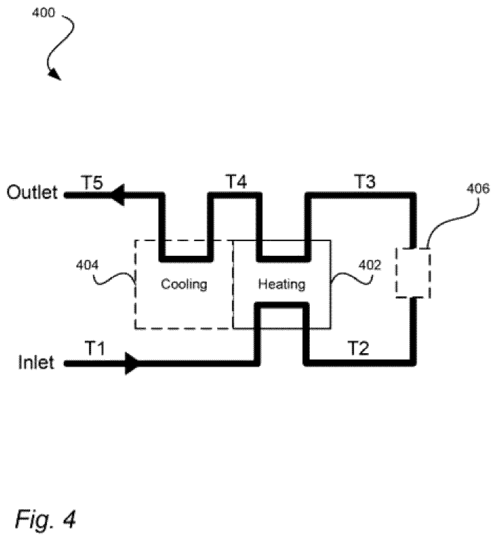

The system 200 further comprises a first heating device 400a. The first heating device 400a may be fluidly connected to the first enzymation section 202. The first heating device 400a may be arranged downstream from the first enzymation section 202. Thus, the first heat exchanger 400a may receive the enzymised oat derived material outputted from the first enzymation section 202. The first heating device 400a may heat the oat derived material 210 from the first enzymation section 202. The first heating device 400a may heat the oat derived material from a first temperature T to a second temperature T′. The first heating device 400a may heat the oat derived material to terminate the enzymation process. The first heating device 400a is further described in connection to FIG. 4.

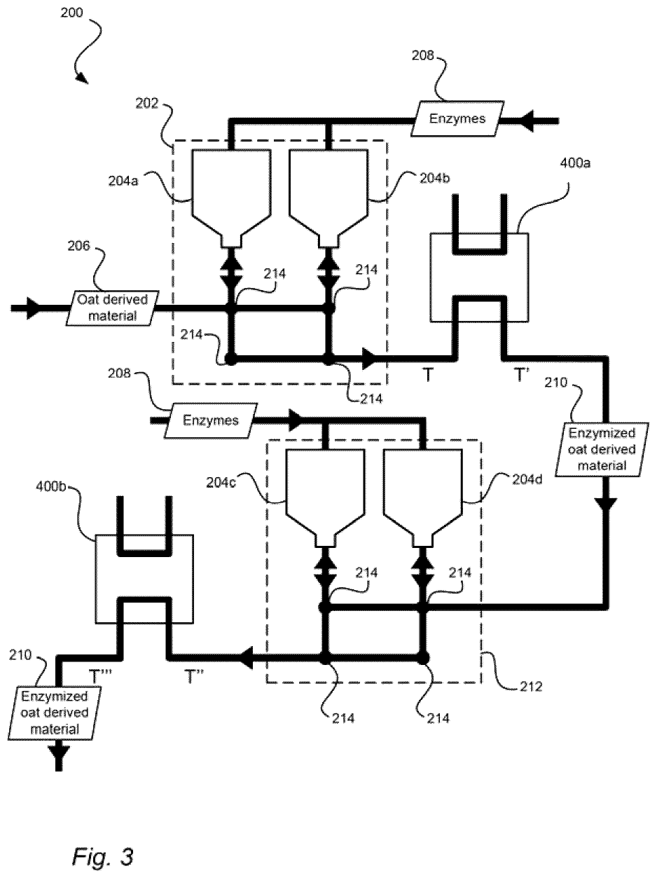

FIG. 3 schematically illustrates another example of the system 200 for preparing the liquid oat base or drink from the oat derived material 206.

In addition to what is described in connection to FIG. 2, the system 200 illustrated in FIG. 3 may further comprise a second enzymation section 212. The second enzymation section 212 is adapted for a second enzymation of the oat derived material 210. The second enzymation section 212 is fluidly connected to the first heating device 400a. The second enzymation section 212 is arranged downstream of the first heating device 400a. The second enzymation section 212 is thus configured to receive oat derived material 210 from the first heating device 400a and enzyme 208 from a supply of enzymes (not shown). Even though the inlet of the enzyme 208 is illustrated as separate from the enzymised oat derived material 210, they may be combined. Put differently, the enzymes 208 may be mixed with the oat derived material prior to entering the second enzymation section 212.

The second enzymation section 212 outputs further enzymised oat derived material 210. The enzymised oat derived material 210 outputted from the second enzymation section 212 may be enzymised to a higher degree than the enzymised oat derived material outputted from the first enzymation section 202. Alternatively, the enzymised oat derived material outputted from the second enzymation section 212 may be enzymised in a different way from the enzymised oat derived material outputted from the first enzymation section 202.

The second enzymation section 212 comprises two enzymation tanks 204. The setup and benefits of the first enzymation section 202 may be the same for this second enzymation section 212 such as for example, the second enzymation section 212 may comprise more than two enzymation tanks 204, such as three, four or more. The at least two enzymation tanks 204 are fluidly connected in parallel. The at least two enzymation tanks 204 be configured to produce a continuous flow of enzymised oat derived material 210. In this illustration, the two enzymation tanks 204 are connected in connection points 214 which may comprise valves, such that one of the enzymation tanks 204 may be emptied, while the other enzymation tank 204 is filled with the oat derived material 206 and enzymes 208 or while the enzymation is taking place. In other words, the two or more enzymation tanks 204c, 204d of the second enzymation section 212 may work together to produce the continuous flow of enzymised oat derived material in the same way as described above in connection with the first enzymation section 202. The volume V, filling time t1, emptying time t2 and enzymation time te of the enzymation tanks 204c, 204d of the second enzymation section 212 may be the same or different as the first enzymation section 202. For example, the volume V of the enzymation tanks 204c, 204d of the second enzymation section 212 may be 6 000 L-14 000 L. How the at least two enzymation tanks 204c, 204d may be connected is further discussed in connection with FIG. 5.

The system 200 may further comprise a second heating device 400b. The second heating device 400b may be fluidly connected to the second enzymation section 212. The second heating device 400b is arranged downstream from the second enzymation section 212. Thus, the second heat exchanger 400b receives the enzymised oat derived material 210 outputted from the second enzymation section 212. The second heating device 400b heats the oat derived material 210 from the second enzymation section 212. The second heating device 400b may heat the oat derived material from a third temperature T″ to a fourth temperature T′″. The second heating device 400b may heat the oat derived material to terminate the enzymation process. The second heating device 400b is further described in connection to FIG. 4.

FIG. 4 schematically illustrates the heating device 400 in the system 200 for preparing the liquid oat base or drink from the oat derived material. The heating device 400 illustrated herein may thus constitute the first heating device 400a and/or second heating device 400b illustrated in FIG. 2 and/or FIG. 3. The heating device 400 may change the temperature of the oat derived material from a temperature T1 to a temperature T5. The temperature T5 may be higher than the temperature T1. Alternatively, the temperature T5 may be the same as the temperature T1. Alternatively, the temperature T5 may be lower than the temperature T1.

The heating device 400 may comprise a heat exchanger 402. The heat exchanger may increase the temperature of the oat derived material from the temperature T1 to a temperature T2. oat derived material. The heating device 400 may further comprise a cooling section 404. The cooling section 404 may be part of the heat exchanger 402, or separate. Thus, the heating device 400 may be adapted to both heat the oat derived material and cool the oat derived material. The cooling section 404 may reduce the temperature of the oat derived material from the temperature T4 to the temperature T5. Thus, even though the temperature T1 of the inputted oat derived material may be the same as, or lower than, the temperature T5 of the outputted oat derived material, the oat derived material may still have been heated to a higher temperature as some point thus terminating the enzymation of the oat derived material.

The heating device 400 may further comprise a steam injector 406. The steam injector 406 may be connected to the heat exchanger 402 and arranged downstream from the temperature change T1 to T2 of the heat exchanger 402 but before the change of the temperature T3 to T4 of the heat exchanger 402. The steam injector 406 may be adapted to steam the oat derived material, thus increasing the temperature of the oat derived material from the temperature T2 to the temperature T3.

As illustrates herein, the heat exchanger 402 may be arranged to exchange heat between the oat derived material before the steam injector 406 and the oat derived material after the steam injector 406. This may be advantageous in that heat energy produced by the steam injector can be re-used in the heating and/or cooling of the oat derived material from the temperature T1 to the temperature T2 and/or T4 to T5, thus saving energy. Further, the oat derived material from the steam injector 406 can be cooled from the temperature T3 to the temperature T4, thus reducing the amount of cooling needed by the cooling section 404.

The heating device 400 is not limited to comprising a heat exchanger and/or steam injector 406. The heating device 400 may be any device suitable for the cause. For example, the heating device 400 may comprise a device for heating the oat derived material by using microwaves. The heating device 400 may be an immersion heater. The heating device may comprise tubular heating elements. The heating device 400 may be a circulation heater. The heating device 400 may be an electrode heater.

The cooling section 404 may be a heat exchanger connected to a cooling medium.

The heating process may take approximately 3-10 minutes such as 3-7 minutes. The time of the heating process in the first and/or second heating device 400a, 400b may be the same or different.

The temperatures T1-T5 may be different in the first heating device 400a and the second heating device 400b.

For example, in the first heating device 400a, the temperature T5 may be 60 degrees C. The temperature T5 may correspond to the second temperature T′ as illustrated in FIG. 2. The temperature T3 may be 110 degrees C.

For example, in the second heating device 400b, the temperature T5 may be 5 degrees C. The temperature T5 may in this case correspond to the fourth temperature T′″ as illustrated in FIG. 3. The temperature T3 may be 90 degrees C.

The Temperatures T1-T5 may be selected based on for example a desired enzyme activation and/or deactivation such as the starch degrading enzyme and/or the protein solubilizing enzyme, and the temperatures T1-T5 may be selected as, Temperature T1 may be 55 to 65 degrees, or more preferably 60 degrees. Temperature T2 may be 75 to 85 degrees, or more preferably 80 degrees. Temperature T3 may be 85 to 95 degrees, or more preferably 90 degrees. Temperature T4 may be 65 to 75 degrees, or more preferably 70 degrees. Temperature T5 may be 55 to 65 degrees, or more preferably 60 degrees.

FIGS. 5a to 5c schematically illustrates some examples of how the enzymation tanks 204 of the enzymation section 202, 212 may be fluidly connected in parallel.

FIG. 5a schematically illustrates the connection shown in FIG. 2 and FIG. 3. In this example, the enzyme 208 and the oat derived material 206 are filled into the enzymation tanks 204 in separate pipes. The inlet for the oat derived material 206 may be the same as the outlet for the enzymised oat derived material 210. The inlet for the oat derived material 206 and the outlet for the enzymised oat derived material 210 may comprise a number of product pipes connected in a number of connection points 214, herein illustrated as four connection points. The connection points may be controlled by one or more valves. The connection points may for example be T-connections or Y-connections. The four connection points may allow for one of the enzymation tanks 204 to be filled, while the other enzymation tank 204 is emptied, without interfering with each other.

In FIG. 5b, another example of the parallel connection between the two enzymation tanks 204 is shown. Different from FIG. 5a is that only a single connection point 214 fluidly connects the flow of oat derived material 206 and enzymised oat derived material 210. Even though not illustrated, the enzymes 208 may be supplied from the same supply of enzymes. The connection point 214 may comprise one or more valves for controlling the flow to the two enzymation tanks 204.

In FIG. 5c, yet another example is shown. Herein, the inlet of enzymes 208 and oat derived material 206 are separated from the outlet of enzymised oat derived material 210. A connection point 214 connects the outlet of the enzymation tanks 204. Even though the inlet of the enzymes 208 and the oat derived material 206 are illustrates as being separate, they may be mixed prior to the enzymation tanks 204 such that the enzymation tanks 204 only have one inlet each.

It should be realized that the illustrations in FIG. 5a to 5c are non-limiting examples which are simplified for illustrative purposes. The scale and size of the different components may not be representative of the reality. Further, points where the product lines connect to the enzymation tanks 204 are not limited to those illustrates herein. For example, even though a line is illustrates as connecting at the top of the enzymation tank 204, it may connect to the enzymation tank 204 at other points as well.

Reference is hereby made to a number of related applications with the same applicant and filing date as the present application. A related application titled A TWO-STEP PROCESS AND SYSTEM FOR PREPARING A LIQUID OAT BASE OR DRINK FROM AN OAT DERIVED MATERIAL discloses a process and system for preparing a liquid oat base or drink from an oat derived material in a two-step enzymation process similar to the herein discussed process. Another related application titled A CONTINUOUS PROCESS AND SYSTEM FOR PREPARING A LIQUID OAT BASE OR DRINK FROM AN OAT DERIVED MATERIAL discloses a process and system for preparing a liquid oat base or drink from an oat derived material where the first and second enzymation sections comprises a first and second volume respectively with continuous inlet and outlet. Another related application titled A MIXED PROCESS AND SYSTEM FOR PREPARING A LIQUID OAT BASE OR DRINK FROM AN OAT DERIVED MATERIAL discloses a process and system for preparing a liquid oat base or drink from an oat derived material where one of the first and second enzymation sections comprises two or more enzymation tanks and the other of the first and second enzymation section comprises a volume with continuous inlet and outlet.

It is understood that other variations in the present invention are contemplated and, in some instances, some features of the invention can be employed without a corresponding use of other features. Accordingly, it is appropriate that the appended claims be construed broadly in a manner consistent with the scope of the invention.

1. A process (S100) for preparing a liquid oat base or drink from an oat derived material, comprising:
filling (S102) a first enzymation tank (204a) with the oat derived material (206) and an enzyme (208),
enzymating (S104) the oat derived material (206) in the first enzymation tank (204a),
filling (S106) a second enzymation tank (204b) with the oat derived material (206) and an enzyme (208), the second enzymation tank (204b) being fluidly connected in parallel with the first enzymation tank (204a),
enzymating (S108) the oat derived material (206) in the second enzymation tank (204b),
producing (S110) a continuous flow of enzymised oat derived material (210) by alternately emptying the first and second enzymation tanks (204a, 204b), and
heating (S112), by a first heating device (400a) fluidly connected to and arranged downstream of the first and second enzymation tanks (204a, 204b), the continuous flow of enzymised oat derived material (210) to terminate the enzymation thereof.

2. The process (100) according to claim 1, further comprising cooling (S116) the enzymised oat derived material (210) after heating (S112) the continuous flow of enzymised oat derived material (210).

3. The process (100) according to claim 2, further comprising steaming (S114) enzymised oat derived material (210) after the step of heating (S112) the enzymised oat derived material (210) to terminate the enzymation and before the step of cooling (S116) the enzymised oat derived material (210).

4. The process (100) according to claim 1, further comprising:
filling (S118) a third enzymation tank (204c) with the enzymised oat derived material (210) and enzymes (208),
enzymating (S120) the enzymised oat derived material (210) in the third enzymation tank (204c),
filling (S122) a fourth enzymation tank (204d) with the enzymised oat derived material (210) and enzymes (208), the fourth enzymation tank (204d) being fluidly connected in parallel with the third enzymation tank (204c),
enzymating (S124) the enzymated oat derived material (210) in the fourth enzymation tank (204d), and
producing (S126) a continuous flow of enzymised oat derived material (210) by alternately emptying the third and fourth enzymation tanks (204c, 204d).

5. The process (100) according to claim 4, further comprising heating (S128), by a second heating device (400b) fluidly connected to and arranged downstream of the third and fourth enzymation tanks (204c, 204d), the continuous flow of enzymised oat derived material (210) a second time to terminate the enzymation thereof.

6. The process (100) according to claim 5, further comprising cooling (132) the enzymised oat derived material (210) a second time after heating (S128) the continuous flow of enzymised oat derived material (210) the second time.

7. The process (100) according to claim 6, further comprising steaming (S130) the enzymised oat derived material (210) a second time after the step of heating (S128) the enzymised oat derived material (210) the second time to terminate the enzymation and before the step of cooling (S132) the enzymised oat derived material the second time.

8. The process (100) according to claim 1, wherein the oat derived material (206) comprises micronized oats or micronized oat-kernels, or oat-flour, or fractions of oat.

9. The process (100) according to claim 1, wherein the oat derived material (206) is provided as an aqueous or water suspension or slurry.

10. The process (100) according to claim 1, wherein the step of enzymating in the first and second enzymation tanks (S104, S108) and/or in the third and fourth enzymation tanks (S120, S124) comprises contacting the oat derived material with starch degrading enzymes, preferably amylase.

11. The process (100) according to claim 1, wherein the step of enzymating in the first and second enzymation tanks (S104, S108) and/or in the third and fourth enzymation tanks (S120, S124) comprises contacting the oat derived material with protein solubilizing enzyme, preferably protein deamidase or protein glutaminase.

12. A system (200) for preparing a liquid oat base or drink from an oat derived material (206), comprising:
a first enzymation section (202) adapted for a first enzymation of the oat derived material (206), and
a first heating device (400a) fluidly connected to and arranged downstream of the first enzymation section (202) and adapted to heat the oat derived material from the first enzymation,
wherein the first enzymation section (202) comprises at least two enzymation tanks (204a, 204b) fluidly connected in parallel, the at least two enzymation tanks (204a, 204b) being configured to produce a continuous flow of enzymised oat derived material (210).

13. The system (200) according to claim 12, wherein the heating device (400a) is adapted to both heat the enzymised oat derived material (210) in a first step and thereafter cool the enzymised oat derived material (210) in a second step.

14. The system (200) according to claim 12, wherein the heating device (400a) comprises a heat exchanger (402) and/or a steam injector (406) oat derived material

15. The system (200) according to claim 12, further comprising,
a second enzymation section (212) adapted for a second enzymation of the oat derived material (210) and fluidly connected downstream to the first heating device (400a), and
a second heating device (400b) fluidly connected to and arranged downstream of the second enzymation section (212) and adapted to heat the oat derived material (210) from the second enzymation,
wherein the second enzymation section (212) comprises at least two enzymation tanks (204c, 204d) fluidly connected in parallel, the at least two enzymation tanks (204c, 204d) being configured to produce a continuous flow of enzymised oat derived material (210).

16. The system (200) according to claim 15, wherein the second heating device (400b) is adapted to both heat the enzymised oat derived material (210) in a first step and thereafter cool the enzymised oat derived material (210) in a second step.

17. The system (200) according to claim 15, wherein the second heating device (400b) comprises a heat exchanger (402) and/or a steam injector (406). oat derived material

18. The system (200) according to claim 12, wherein the first enzymation section (202) and the second enzymation section (212) comprises a conduit or a reservoir, such as a tank, respectively.
